(19) United States
(12) Patent Application Publication
(10) Pub. No.: US 2024/0382219 A1
(43) Pub. Date: Nov. 21, 2024

(54) TEMPLATE FOR TIBIAL BASEPLATE REMOVAL

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Roger Ryan Dees, JR., Drummonds, TN (US); Forrest A. James, Memphis, TN (US); Brian W. McKinnon, Arlington, TN (US); Timothy J. Bourne, Memphis, TN (US)

(73) Assignees: Smith & Nephew, Inc., Memphis, TN (US); Smith & Nephew Orthopaedics AG, Zug (CH); Smith & Nephew Asia Pacific PTE. Limited, (omitted)

(21) Appl. No.: 18/655,704

(22) Filed: May 6, 2024

Related U.S. Application Data
(60) Provisional application No. 63/466,405, filed on May 15, 2023.

(51) Int. Cl. A61B 17/16

(52) U.S. Cl. CPC A61B 17/1675

(57) ABSTRACT

An orthopedic template configured to assist surgeons in removing a tibial implant previously implanted within a patient's bone. In use, the template provides a reference that mimics the underlying support member of the previously implanted tibial implant. That is, the template provides a representation or footprint that mimics the configuration of the underlying support member of a tibial implant, which cannot be readily seen when the tibial implant is inserted into a patient's bone. For example, the template may include a superiorly extending structure or a visual image that mimics the underlying configuration of the support member of the tibial implant. In use, the template may be positioned on a superior surface of the tibial implant. Thereafter, an instrument such as a bone cutter, can be used to trace the representation formed on the template while simultaneously cutting or removing bone beneath the tibial tray.

### CROSS-REFERENCE TO RELATED APPLICATIONS

This is a non-provisional of, and claims the benefit of the filing date of, U.S. provisional patent application No. 63/466,405, filed May 15, 2023, entitled “Template for Tibial Baseplate Removal,” the entirety of which application is incorporated by reference herein.

### FIELD OF THE DISCLOSURE

The present disclosure is directed to a device, system, and/or method for removing an orthopedic implant, and more particularly to a template, and corresponding tools and methods, for removing a previously implanted tibial implant.

### BACKGROUND

Knee arthroplasty or knee replacement procedures generally involve the implantation, installation, etc. (used interchangeably herein without the intent to limit or distinguish) of an orthopedic implant such as a knee implant onto a patient's knee. For example, in connection with a total knee replacement, the orthopedic implant (e.g., knee implant) may include a femoral implant and a tibial implant. In use, the femoral implant is attached to the patient's femur while the tibial implant is attached to the patient's tibia. Generally speaking, the femoral and tibial implants may each include a support member (e.g., an intramedullary stem), which is attachable to a load bearing component, an articular surface in connection with a femoral implant, or a tray or baseplate in connection with a tibial implant (terms used interchangeably herein without the intent to limit or distinguish). In use, the support member is arranged and configured to be inserted within an intramedullary canal of the patient's bone while, for example, the load bearing component resides upon a prepared surface on the patient's bone. A bearing member or insert is typically mounted upon the tibial tray of the tibial implant and resides between the tibial implant and the femoral implant.

Recently, with the advent of modern-day manufacturing (e.g., additive manufacturing, three-dimensional printing, selective laser sintering (SLS), direct metal laser sintering (DMLS), electron beam melting (EBM), selective laser melting (SLM), etc.), stem components having increasingly complex shapes have been developed to, for example, improve rotational stability and/or fixation of the implant (e.g., tibial implant) to the patient's bone, to provide improved load distribution, etc.

One potential disadvantage of these increasingly complex shapes however is that during a revision surgery, one or more previously implanted orthopedic implants needs to be removed. In doing so, the surgeon is tasked with removing the implant, for example, the tibial implant. However, during a revision surgery, the stem component of the tibial implant cannot be seen by the surgeon since the stem component is implanted within the intramedullary canal of the patient's bone and the inferior features of the tibial implant are covered by the tibial tray. Thus, surgeons are tasked with removing an implant implanted within a patient's bone thereby making it such that portions of the implant cannot be seen, while attempting to maintain as much of the patient's existing bone as possible and while attempting to not contact the stem component, which may lead to metal debris.

Currently, to remove a tibial implant, surgeons often utilize osteotomes, saws, chisels, etc. to cut away the patient's bone incrementally and blindly adjacent the interface with the tibial implant. This task is rendered increasing difficult since the specific features or configuration of the previously implanted tibial implant cannot be seen. For example, the surgeon cannot see the specific keels, fins, pegs, etc. of the stem component of the tibial implant. This is becoming increasingly troublesome as the implant configurations become increasingly complex.

It is with this in mind that the present disclosure is provided.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

In accordance with one or more features of the present disclosure, an orthopedic template is disclosed. In use, the orthopedic template is arranged and configured to assist with removing a previously implanted orthopedic tibial implant during a reversion surgery.

In some examples, the template includes a superior surface and an inferior surface opposite the superior surface, the superior surface including a footprint or representation mimicking a configuration of an underlying support member of the tibial implant.

In any preceding or subsequent example, the footprint or representation is arranged and configured to interact with a first tip of an instrument such that moving the first tip of the instrument relative to the footprint or representation causes a second tip of the instrument to cut bone located inferiorly of the tibial implant.

In any preceding or subsequent example, the footprint or representation extends superiorly from the superior surface of the template.

In any preceding or subsequent example, the footprint or representation is a three-dimensional structure extending from the superior surface of the template.

In any preceding or subsequent example, the footprint or representation is a printed image that mimics the configuration of the support member of the tibial implant.

In any preceding or subsequent example, the template is arranged and configured to be positioned on a superior surface of the tibial implant.

In any preceding or subsequent example, the template is coupled to the tibial implant.

In any preceding or subsequent example, the template includes an outer shape that mimics an outer shape of the tibial implant.

In accordance with one or more features of the present disclosure, a system for assisting with removing a previously implanted orthopedic tibial implant during a reversion surgery is disclosed. In some examples, the system includes a tibial implant and a corresponding orthopedic implant. The tibial implant including a tibial tray having a superior surface and an inferior surface, and a support member extending from the inferior surface of the tibial tray, the support member including a configuration that includes an intramedullary stem arranged and configured for implantation into an intramedullary canal of a patient's bone, and at least one of: one or more fins extending from the intramedullary stem, one or more keels extending from the intramedullary stem, one or more ridges extending along a length of the one or more keels, one or more pegs extending from the inferior surface of the tibial tray, one or more bridge members coupling the one or more pegs to the one or more keels, respectively, or a combination thereof. The orthopedic template includes a superior surface and an inferior surface opposite the superior surface, the template being arranged and configured to be positioned on the superior surface of the tibial tray, the superior surface of the template including a footprint or representation mimicking the configuration of the support member of the tibial implant.

In any preceding or subsequent example, the system further includes an instrument including a distal end having first and second tips; wherein the footprint or representation formed on the superior surface of the template is arranged and configured to interact with the first tip such that moving the first tip of the instrument relative to the footprint or representation causes the second tip to cut bone located inferiorly of the tibial implant.

In accordance with one or more features of the present disclosure, a system arranged and configured to assist with removing a previously implanted orthopedic tibial implant during a reversion surgery is disclosed. The tibial implant including a tibial tray and a support member, the tibial tray including a superior surface and an inferior surface, the support member extending inferiorly from the inferior surface of the tibial tray. The system including an orthopedic template and an instrument.

In some examples, the template includes a superior surface and an inferior surface opposite the superior surface, the template being arranged and configured to be positioned on the superior surface of the tibial tray, the superior surface of the template including a footprint or representation mimicking a configuration of the support member of the tibial implant.

In some examples, the instrument includes a distal end having first and second tips, wherein the first tip is arranged and configured to interact with the footprint or representation formed on the superior surface of the template such that moving the first tip of the instrument relative to the footprint or representation causes the second tip to cut bone located inferiorly of the tibial implant.

In some of the preceding or subsequent examples, the first tip has a first length and the second tip has a second length smaller than the first length.

In some of the preceding or subsequent examples, the first and second lengths are configured so that when the first tip contacts the footprint or representation on the template, the second tip approaches but does not contact the support member of the tibial implant.

In any preceding or subsequent example, the footprint or representation extends superiorly from the superior surface of the template.

In any preceding or subsequent example, the footprint or representation is a three-dimensional structure extending from the superior surface of the template.

In any preceding or subsequent example, the footprint or representation is a printed image that mimics the configuration of the support member of the tibial implant.

In any preceding or subsequent example, the template is coupled to the tibial implant.

In some of the preceding or subsequent examples, the template is a sticker. For example, the sticker may be arranged and configured to be directly adhered to the superior surface of the tibial implant. Alternatively, the sticker may be adhered to the superior surface of the template, which may then be coupled to the superior surface of the tibial implant.

In accordance with one or more features of the present disclosure, a method for removing a tibial implant previously implanted within a patient's bone is disclosed. In use, the tibial implant includes a tibial tray and a support member extending inferiorly of the support member, the support member having a configuration. In some examples, the method includes positioning an orthopedic template on a superior surface of the tibial implant, the template including a superior surface having a footprint or representation mimicking a configuration of a support member of the tibial implant; tracing, using a first tip of an instrument, the footprint or representation formed on the superior surface of the template so that a second tip of the instrument cuts bone positioned inferiorly of the tibial tray; and removing the tibial implant from the patient's bone.

Examples of the present disclosure provide numerous advantages. For example, by providing a template including a footprint mimicking the underlying support member of a tibial implant, surgeons are able to utilize the template to trace the outline of the underlying support member thereby assisting surgeons with removing the tibial implant from the patient's bone.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1A is a front view of an example of a conventional orthopedic tibial implant;

FIG. 1B is a bottom, perspective view of the tibial implant shown in FIG. 1A;

FIG. 1C is a bottom view of the tibial implant shown in FIG. 1A;

FIG. 1D illustrates a perspective view of an example of a prepared tibial, the prepared tibial being arranged and configured to receive the tibial implant shown in FIG. 1A;

FIG. 2A illustrates a top, perspective view of a template in accordance with one or more features of the present disclosure, the template being arranged and configured to be used with the tibial implant shown in FIG. 1A during a revision procedure;

FIG. 2B is a bottom, perspective view of the template shown in FIG. 2A;

FIG. 2C is a top view of the template shown in FIG. 2A;

FIG. 3A illustrates a top, perspective view of the template shown in FIG. 2A coupled to the tibial implant shown in FIG. 1A;

FIG. 3B illustrates a top, perspective view of an example of an instrument or bone cutter in accordance with one or more features of the present disclosure being used in a procedure with the template and tibial implant of FIG. 3A to remove bone adjacent the tibial implant;

FIG. 3C illustrates a top view of the instrument or bone cutter being used in a procedure with the template and tibial implant of FIG. 3A;

FIG. 3D illustrates a detailed, perspective view of the instrument or bone cutter shown in FIG. 3B;

FIG. 3E illustrates a cross-sectional view of the instrument or bone cutter being used in a procedure with the template and tibial implant of FIG. 3A; and

FIG. 3F illustrates a cross-sectional view of the instrument or bone cutter shown in FIG. 3B.

The drawings are not necessarily to scale. The drawings are merely representations, not intended to portray specific parameters of the disclosure. The drawings are intended to depict examples of the disclosure, and therefore are not to be considered as limiting in scope. In the drawings, like numbering represents like elements.

### DETAILED DESCRIPTION

Various features or the like of an orthopedic device or template (terms used interchangeably herein without the intent to limit or distinguish), and corresponding tools, systems, and methods, will now be described more fully hereinafter with reference to the accompanying drawings, in which one or more features of the orthopedic template and corresponding tools, systems, and methods, will be shown and described. It should be appreciated that the various features or the like may be used independently of, or in combination, with each other. It will be appreciated that an orthopedic template as disclosed herein may be embodied in many different forms and should not be construed as being limited to the examples set forth herein. Rather, these examples are provided so that this disclosure will convey certain features of the orthopedic template and corresponding tools, systems, and methods to those skilled in the art.

As will be described herein, in accordance with one or more features of the present disclosure, an orthopedic template is disclosed. In use, the orthopedic template is arranged and configured to provide a reference that mimics the previously implanted knee prosthesis such as, for example, the tibial implant. That is, in some examples, the orthopedic template includes and provides a reference or footprint that mimics the configuration of the underlying support member of a tibial implant, which cannot be readily seen when the tibial implant is inserted into a patient's bone. For example, the template may provide a footprint in the form of a superiorly extending structure such as, for example, a three-dimensional printed structure, that mimics the underlying configuration of the inferior features (e.g., support member) of the tibial implant. Alternatively, the template may provide a footprint in the form of a visual image (e.g., printed image, a laser etched image, etc.) that mimics the underlying configuration of the inferior features (e.g., support member) of the tibial implant. In either scenario, in use, the orthopedic template may be positioned on a first, top, or superior surface of the tibial tray to provide a surgeon with an improved visual reference or footprint of the underlying inferior features of the previously implanted tibial implant, which cannot be seen. Collectively both the visual image and/or the superiorly extending structure are referred to herein as a footprint.

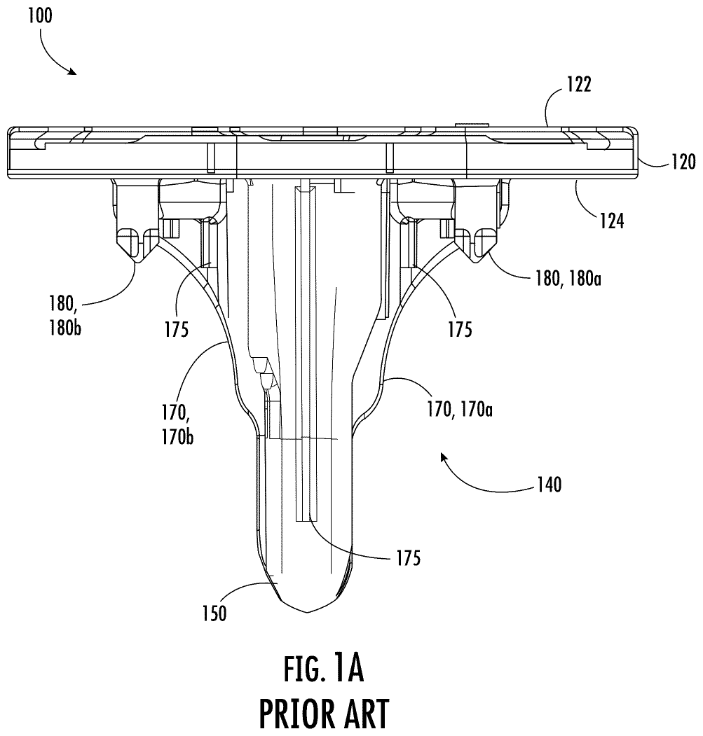

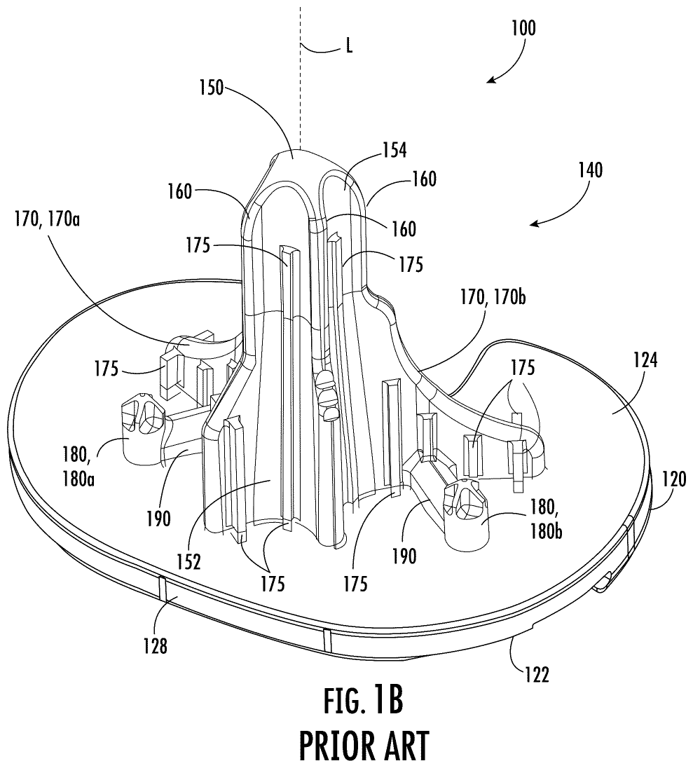

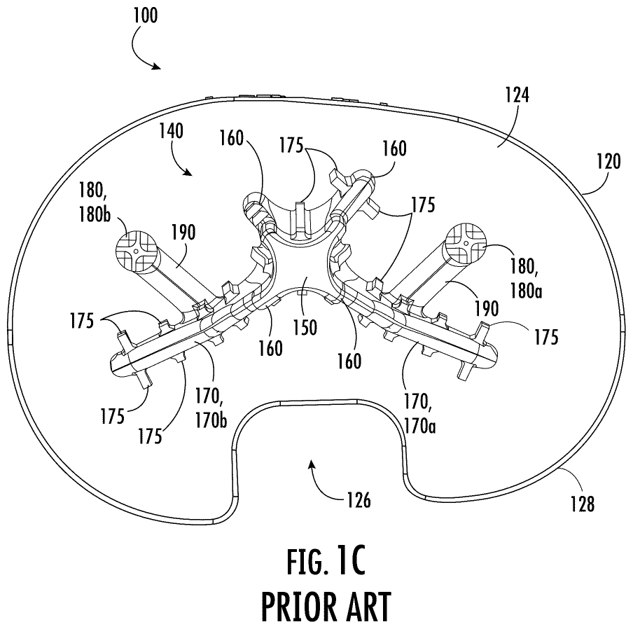

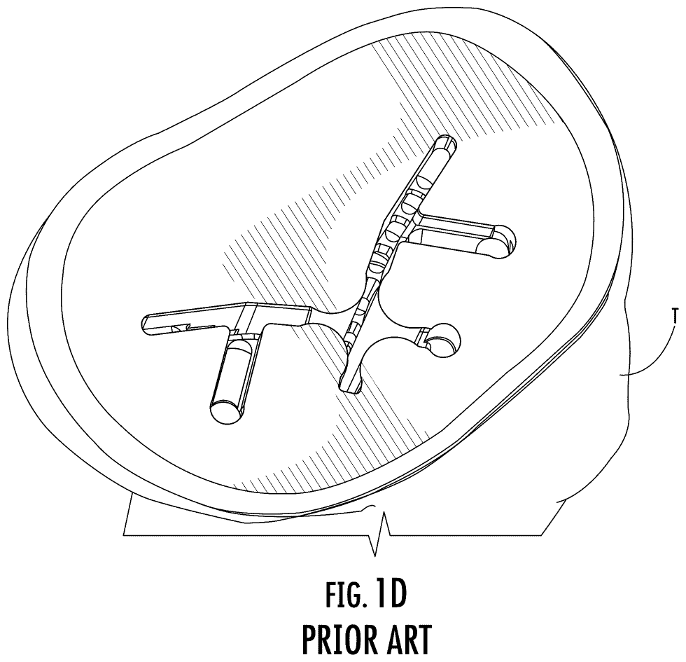

Referring to FIGS. 1A-1C, an example of a tibial implant 100 is illustrated. As illustrated in the example shown, the tibial implant 100 includes a tibial tray 120 connected to a support member 140 including an intramedullary stem or stem portion 150 (terms used interchangeably herein without the intent to limit or distinguish) arranged and configured for implantation into an intramedullary canal of a patient's bone such as, for example, the patient's tibia. The tibial tray 120 includes a first, top, or superior surface 122 and a second, bottom, or inferior surface 124 (terms used interchangeably herein without the intent to limit or distinguish). The inferior surface 124 of the tibial tray 120 may include a porous layer or coating (not shown) applied thereto. In addition, in some examples, the tibial tray 120 may include a posterior notch 126, which may be designed to allow preservation of the attachment site of a posterior cruciate ligament, although, in other examples, the tibial tray 120 may or may not include this or other notches or gaps for preserving one or both of the cruciate ligaments.

The support member 140 extends from the inferior surface 124 of the tibial tray 120. In use, and as will be readily understood by one of ordinary skill in the art, the support member 140 is positioned, at least partially, within the intramedullary canal of the patient's tibia T (FIG. 1D) to couple the tibial implant 100 to the patient's tibia T. In some examples, as illustrated, the support member 140 may include a stem portion 150 that extends away from the inferior surface 124 of the tibial tray 120 along a longitudinal axis L. In some examples, the longitudinal axis L may be substantially perpendicular to the inferior surface 124 of the tibial tray 120, although this is but one configuration and the longitudinal axis L of the stem portion 150 may be angled relative to the inferior surface 124 of the tibial tray 120. In addition, and/or alternatively, the stem portion 150 may be positioned offset from a center of the tibial tray 120 (e.g., positioned more medial than lateral). For example, the stem portion 150 may be medialized slightly from the center of the tibial tray 120.

The stem portion 150 may have any suitable cross-sectional shape and/or configuration. For example, as illustrated, the stem portion 150 may include a first portion 152 adjacent to the inferior surface 124 of the tibial tray 120 and a second portion 154 that extends away from the first portion 152. The first portion 152 may have a first cross sectional area and the second portion 154 may have a second cross sectional area wherein the first cross sectional area is larger than the second cross sectional area. In other examples, the second portion 154 can have the same cross-sectional area and shape as the first portion 152. In use, the stem portion 150 has a length that is sized to promote varus-valgus stability and resistance of the tibial tray 120 to liftoff from the patient's bone.

In addition, in some examples, the support member 140 may include one or more fins 160 spaced about the stem portion 150. For example, as illustrated, the stem portion 150 may include four fins 160 spaced thereabout, although this is but one configuration and more or less fins 160 may be used. Moreover, in some examples, the fins 160 can be spaced equally from each other or in another arrangement as desired. In some examples, the fins 160 may extend along the support member 140 and may be monolithically formed therewith. In some examples, the fins 160 may extend towards the second portion 154 in a tapering configuration. In use, the fins 160 assist in providing rotational stability and help with implantation and alignment of the tibial tray 120 in bone upon implantation. Any or all of the fins 160 can also be sized to a maximum that is implantable based on the anatomy of the patient.

In addition, the support member 140 may also include one or more posterior keels 170. As shown, the support member 140 may include first and second posterior keels 170a, 170b mounted on the inferior surface 124 of the tibial tray 120 extending from either side of the stem portion 150 posteriorly towards the posterior side P of the tibial tray 120, although this is just one configuration and more or less keels 170 and/or different configurations of keels 170 may be utilized. In use, the keels 170 may increase the strength of the tibial implant 100 while also helping to prevent rotation of the tibial implant 100 relative to the patient's bone (e.g., keels 170 assist with rotational stability of the tibial tray 120 in bone upon implantation).

In some examples, the first keel 170a may be angled relative to the second keel 170b, but in other examples the first and second keels 170a, 170b may be substantially aligned with one another. In some examples, the first and second keels 170a, 170b may be separated from each other via an angle ranging from about 10 degrees to about 180 degrees. As illustrated, in some examples, the first and second keels 170a, 170b may have a similar shape and size, however, in other examples, the first and second keels 170a, 170b may be provided with different shapes and sizes. In some examples, the first and second keels 170a, 170b curve or curl towards the medial M and lateral L sides, respectively, of the tibial tray 120.

The first and second keels 170a, 170b may extend longitudinally along the support member 140 towards the second portion 154; however, as illustrated, the first and second keels 170a, 170b may be shorter or smaller than the fins 160 and, as such, do not extend as far longitudinally as the fins 160 along the support member 140. Each of the first and second keels 170a, 170b may have a horizontal length that extends towards a rim or periphery edge 128 of the tibial tray 120. The first and second keels 170a, 170b may each have a sharp edge.

In addition, in some examples, the keels 170 (e.g., the first and second keels 170a, 170b), as well as other components of the support member 140, may include a plurality of ridges 175 that extend along the height or a portion of the keels 170 as measured along the longitudinal axis L. In some examples, the plurality of ridges 175 may have a square or semi square cross-sectional shape however in other forms the shape may be rectangular or semicircular. In use, the plurality of ridges 175 assist with added bone compression and fixation strength of the tibial implant 100 in the patient's bone.

In addition, as illustrated, in some examples, the tibial tray 120 may also include one or more pegs 180. For example, as shown, the tibial implant 100 may include first and second pegs 180a, 180b, although this is but one configuration and more or less pegs may be used. As illustrated, the first and second pegs 180a, 180b may be mounted on the inferior surface 124 of the tibial tray 120. In some examples, as illustrated, the first and second pegs 180a, 180b may be positioned closer to the rim or periphery edge 128 of the tibial tray 120. The first and second pegs 180a, 180b approach the tibial plateau for added stability of the tibial tray 120 and enter into denser bone than in the central canal upon implantation. Additional pegs 180 can be incorporated as desired. The pegs 180 can have any suitable size and shape arranged and configured to engage the patient's bone to provide increased stability. For example, as shown, the pegs 180 may be substantially cylindrical with a pointed tip, although other shapes are envisioned such as, for example, an arrowhead shape.

In addition, in some examples, the pegs 180 such as, for example, the first and second pegs 180a, 180b may be coupled to the support member 140 such as, for example, to the first and second keels 170a, 170b, respectively, via a support rib, a bridge or a bridge member, a radius, material, or the like 190 (terms used interchangeably herein without the intent to limit). During use, the bridge 190 may be arranged and configured to couple the first and second pegs 180a, 180b to the first and second keels 170a, 170b, respectively, so that any loads received by the first and second pegs 180a, 180b can be transferred to the support member 140 via the first and second keels 170a, 170b.

In use, as illustrated in FIG. 1D, the tibial implant 100 including the inferior surface 124 of the tibial tray 120 may be positioned within or nested into a prepared proximal tibia T of the patient. In use, the patient's proximal tibia T may be prepared using precision milling via, for example, a bur associated with a robotic surgical system, although any other mechanisms for preparing the patient's proximal tibial T may be used.

Additional information on examples of the tibial implant can be found in U.S. patent application Ser. No. 18/009,947, filed on Dec. 12, 2022, entitled “Tibial Implant with Improved Anterior Load Transfer,” the entire contents of said application being incorporated herein.

While a particular example of a tibial implant has been shown and described herein, as will be described in greater detail herein, the present disclosure is directed to devices, systems, and methods arranged and configured to assist surgeons with removing a previously implanted tibial implant as such the present disclosure is not limited to any particular tibial implant unless explicitly claimed. As such, the present disclosure should not be limited to the features of the tibial implant disclosed herein unless explicitly claimed.

Thus described, with the advent of modern manufacturing techniques, tibial implants may include increasingly more complex support members. Such designs provide numerous advantages in, for example, providing increased rotational resistance and strength for the tibial implant when implanted, improved fixation between the tibial implant and the patient's bone post-operatively, improve load-transfer, etc. By varying the configuration of the support member including the configuration of the stem, the fins, the keels, and/or the pegs, numerous differently configured tibial implants can be manufactured.

However, during a revision surgery in which a previously implanted tibial implant must be removed from the patient's bone, such complex designs may hinder a surgeon's ability to remove the previously implanted tibial implant while maintaining as much of the patient's existing bone as possible, especially since the support member cannot be seen once implanted.

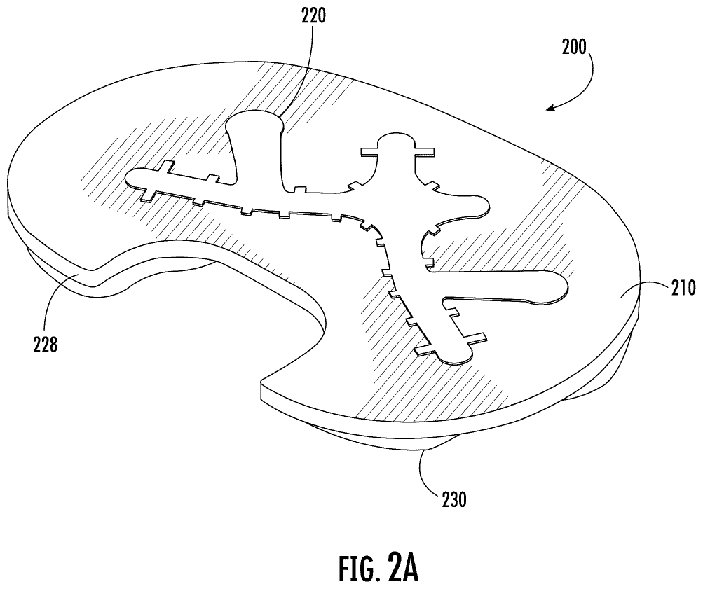

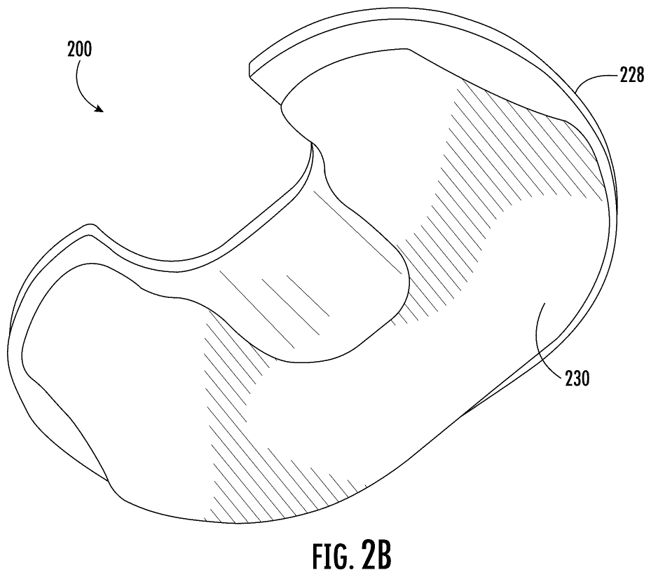

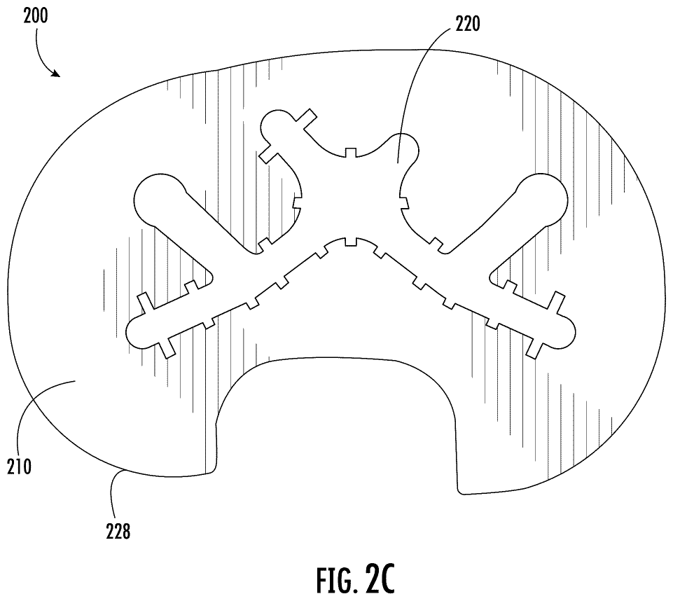

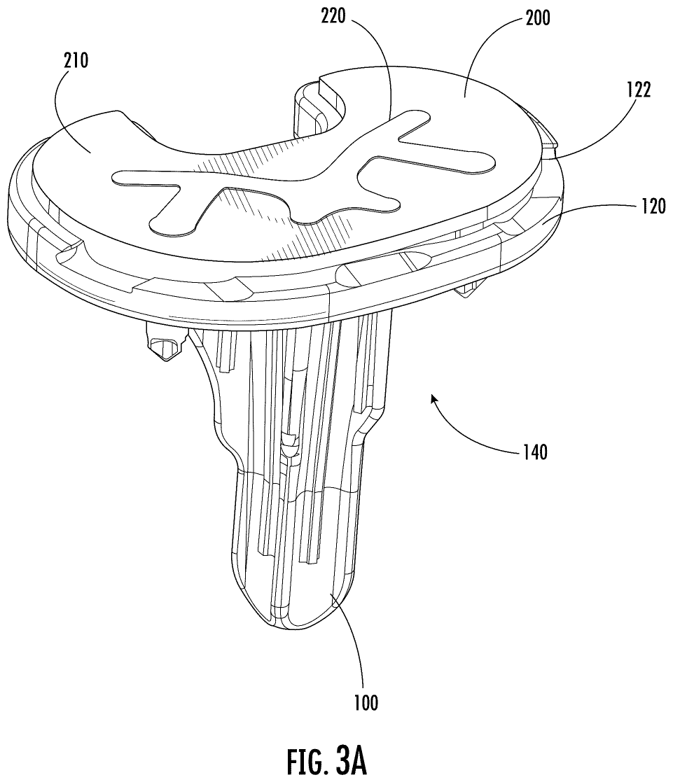

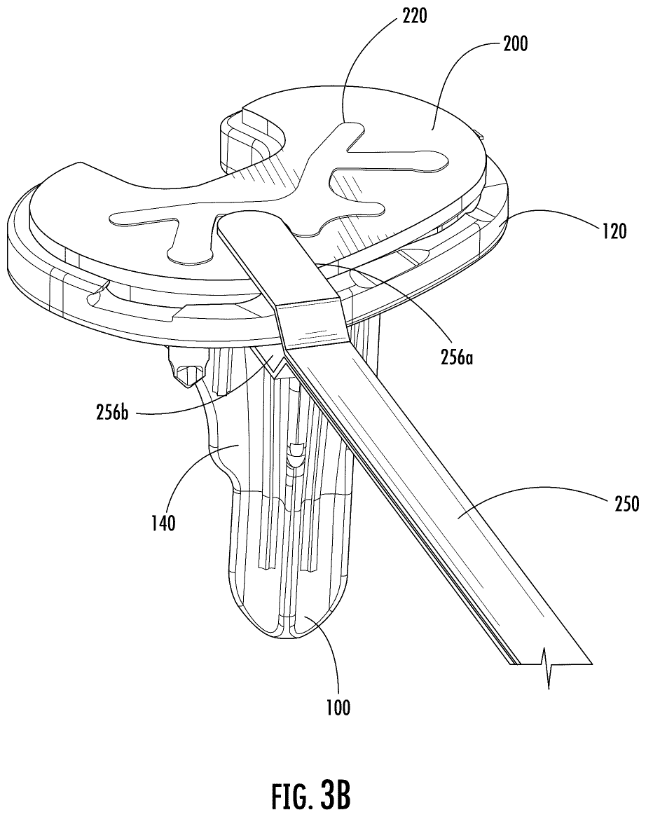

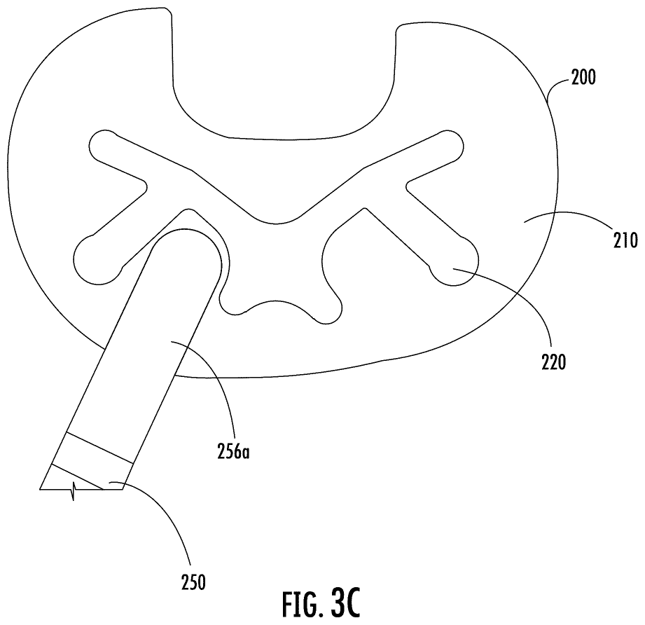

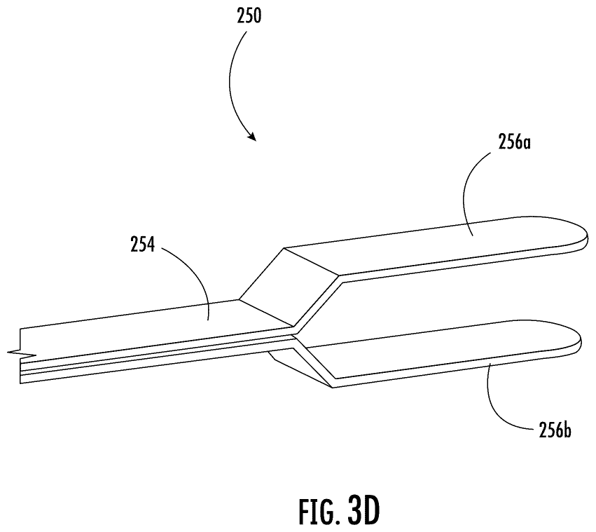

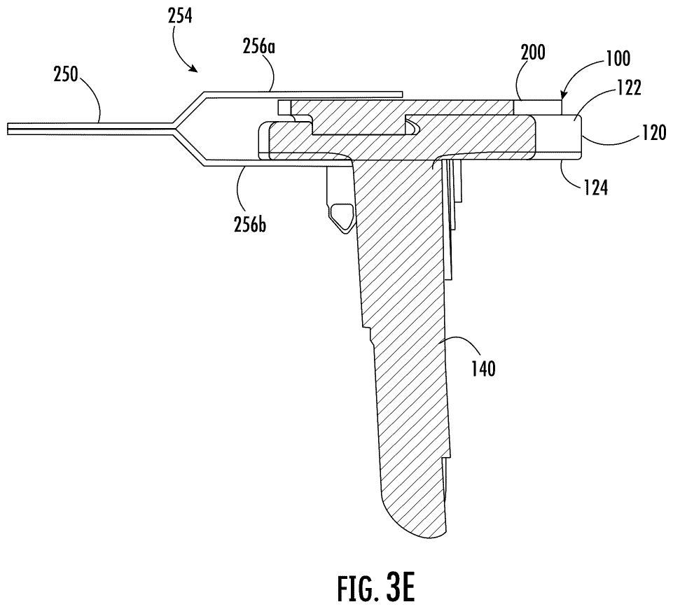

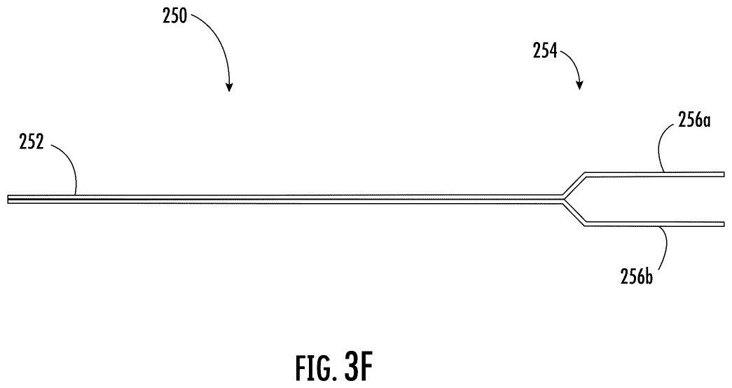

In accordance with one or more features of the present disclosure, with reference to FIGS. 2A-3F, an example of an orthopedic template 200 arranged and configured to assist surgeons with removing a tibial implant such as, for example, tibial implant 100, is disclosed. As illustrated, in some examples, the template 200 includes a first, top, or superior surface 210 (terms used interchangeably herein without the intent to limit or distinguish) and a second, bottom, or inferior surface 230 (terms used interchangeably herein without the intent to limit or distinguish). In use, the template 200 may include an outer shape or periphery rim 228 that mimics the outer shape or periphery edge 128 of the tibial implant 100. In addition, in use, the template 200 is arranged and configured to be positioned on the superior surface 122 of the tibial implant 100. In some examples, the template 200 may be coupled to the superior surface 122 of the tibial implant 100 by any suitable mechanism now known or hereafter developed such as, for example, a press-fit, a snap-fit, adhesive, etc. In some examples, the template 200 may be coupled to the superior surface 122 of the tibial implant 100 using the same system on the tibial tray 120 that is used to couple the insert to the tibial tray 120. For example, the superior surface 122 of the tibial tray 120 may include a lip or lock (not shown) for receiving and/or securing the template 200. Alternatively, in some examples, the template 200 may be in the form of a sticker, which may be adhered or affixed directly to the superior surface 122 of the tibial tray 120 of the tibial implant 100.

In any scenario, as illustrated, the superior surface 210 of the template 200 includes a representation or a footprint 220 mimicking the configuration of the underlying support member of the tibial implant for the corresponding tibial implant for which the template 200 is intended to be used with (e.g., to facilitate removal thereof). In some examples, the footprint 220 may be in the form of a superiorly extending structure such as, for example, a three-dimensional structure extending from the superior surface 210 of the template 200 that mimics the underlying configuration of the support member of the corresponding tibial implant. For example, the footprint 220 may be in the form of a superiorly extending structure such as, for example, a three-dimensional structure, that extends from the superior surface 210 of the template 200 with a height of between 1 mm and 12 mm, preferably in some examples, the footprint 220 extends 5 mm above the superior surface 210 of the template 200. Thus arranged, as will be described in greater detail below, a first tip 256a of a bone cutting instrument can contact the footprint 220.

Alternatively, the footprint 220 may be in the form of a visual image (e.g., a printed image, a laser etched image, etc.) that mimics the underlying configuration of the support member of the corresponding tibial implant. For example, the footprint 220 may be arranged and configured as a sticker, which may be adhered or affixed directly to the superior surface 210 of the template 200.

In any scenario, in use, with the template 200 positioned on the superior surface 122 of the tibial implant 100, the template 200 provides surgeons with a footprint that mimics the inferior features (e.g., support member) of the corresponding tibial implant 100, which cannot be readily seen with the tibial implant 100 inserted into the patient's bone. That is, the template 200 provides surgeons with a reference for the support member 140 of the tibial implant 100, which they cannot see, thereby providing surgeons with increased confidence when cutting the patient's bone.

With reference to FIGS. 3A-3C, with the template 200 positioned on the superior surface 122 of the tibial implant 100, an instrument or stylus (terms used interchangeably herein without the intent to limit or distinguish) such as, for example, a bone cutter, a sharpened tip, a chisel, a saw, an osteotome, or any other suitable bone cutting instrument now known or hereafter developed, can be utilized in combination with the template 200 to assist surgeons to remove bone adjacent the previously implanted tibial implant 100 to facilitate removal of the tibial implant 100 from the patient's bone. For example, as shown, the bone cutter 250 may include a proximal end 252 and a distal end 254. In some examples, the proximal end 252 may be configured as or with a handle. Alternatively, the proximal end 252 may be arranged and configured to be coupled to a bone removal instrument such as, for example, a powered orthopedic instrument arranged and configured to drive or oscillate the bone cutter 250.

In some examples, the distal end 254 may include first and second tips 256a, 256b. In use, the first tip 256a may be arranged and configured to interact with the footprint 220 formed on the superior surface 210 of the template 200, which is positioned on the superior surface 122 of the tibial implant 100. For example, the first tip 256a may be configured with a non-sharpened or non-cutting tip such as, for example, a blunt tip. In use, the first tip 256a can be used to contact, trace, interact with, etc. the footprint 220 formed on the template 200. In use, the first tip 256a acts as a visual representation to visualize the position of the second tip 256b.

The second tip 256b may be arranged and configured with a sharpened or cutting tip designed to cut or remove bone. In use, the second tip 256b is arranged and configured to reside along the inferior surface 124 of the tibial implant 100. Thus arranged, in use, by utilizing the template 200 and moving the first tip 256a of the bone cutter 250 to trace, follow, contact, interact with, etc. the footprint 220 formed on the superior surface 210 of the template 200, the second tip 256b cuts away at the patient's bone adjacent to the surfaces of the tibial implant 100.

That is, in some examples, when utilized with a template 200 including a superiorly extending footprint 220, the template 200 may be positioned on the superior surface 122 of the tibial implant 100. Thereafter, surgeons can manipulate the bone cutter 250 so that the first tip 256a contacts or approaches the footprint 220 formed on the superior surface 210 of the template 200. By moving the first tip 256a of the bone cutter 250 into the various notches, spaces, etc. of the footprint 220 formed on the template 200 (e.g., by tracing the outline of the footprint 220 with the first tip 256a of the bone cutter 250), the second tip 256b is similarly moving along the inferior surface 124 of the tibial implant 100 thereby providing surgeons with a visual guide that the various notches or spaces are being cut. By mimicking the features and/or configuration of the support member 140 of the tibial implant 100 on the superior surface 210 of the template 200, the second tip 256b of the bone cutter 250 is cutting the patient's bone adjacent to the tibial implant 100 thereby facilitating removal of the tibial implant 100 from the patient's bone. Thus arranged, the first tip 256a and footprint 220 act as a visual guide to assist surgeons in removing bone adjacent the support member 140 of a previously implanted tibial implant 100, which they cannot see (e.g., the footprint 220 acts as a visual representation or reference of the support member 140 and the first tip 256a acts as a visual representation or reference for the position of the second tip 256b, which the surgeon cannot see).

In some examples, the first and second tips 256a, 256b may be registered to each other. Alternatively, the first and second tips 256a, 256b may be provided with an axial offset. For example, the first tip 256a may be configured with a longer length than the second tip 256b. Thus arranged, in use, the surgeon may move the first tip 256a into contact with or adjacent to the footprint 220 on the template 200 while being assured that the second tip 256b approaches but does not contact the support member 140 of the tibial implant 100 thereby preventing, or at least minimizing, the risk of damaging and/or chipping the tibial implant 100, which may cause unwanted metal debris. That is, in some examples, the first tip 256a may have a longer length (e.g., extend farther) than the second tip 256b so that, in use, the surgeon is provided with additional assurance that the second bone-cutting tip 256b does not contact the support member 140 of the tibial implant 100 thereby preventing, or at least minimizing, the likelihood that the second bone-cutting tip 256b contacts the support member 140.

Thus arranged, in use, the template 200 provides a visual and/or structural footprint that can be utilized by surgeons to guide an instrument such as, for example, a bone cutter, to identify, locate, and/or remove bone adjacent the support member of the tibial implant, which cannot be readily seen by the surgeon. In some examples, the template can assist surgeons with identifying and locating the stem, the fins, the keels, and/or the pegs. That is, during a revision surgery, a surgeon may position and/or secure a template on the superior surface of the tibial implant. Thereafter, using an instrument, such as, for example, a bone cutter, the surgeon can trace the outline of the footprint provided on the superior surface of the template. The footprint mimicking the support member extending from the inferior surface of the tibial implant. Tracing the footprint of the template with the bone cutter causes a second tip or cutter of the bone cutter, which is positioned inferiorly of the tibial tray, to cut and/or remove bone from the patient's bone adjacent to the implant thereby facilitating removal of the tibial implant from the patient's bone. In some examples, the bone cutter may be arranged and configured to provide surgeons with additional assurances that the bone-cutting tip is not contacting the tibial implant thereby preventing, or at least minimizing, the likelihood of unwanted metal debris.

Alternatively, in some examples, as opposed to providing a visual image or superiorly extending structure, the template may include one or more openings or slots to guide a marker or other marking, engraving, or etching tool. In use, the openings or slots formed in the template can be used to guide a marking tool to scribe, etch, or mark on the superior surface of the tibial tray, which can then be used as a reference (e.g., to mark the inferior tibial implant surface profile in the superior surface of the tibial implant).

The template can be manufactured from any suitable bio-compatible material now known or hereafter developed used including, for example, plastics, mylar, or the like. Alternatively, the template can be manufactured from a metal such as, for example, titanium, cobalt chrome, stainless steel, or a ceramic or other biocompatible material. In addition, and/or alternatively, the template may be formed using any appropriate technology now known or hereafter developed. For example, the template could be 3 D printed or manufactured using any now known or hereafter developed additive manufacturing technique.

Terms such as top, bottom, superior, inferior, medial, lateral, anterior, posterior, proximal, distal, and the like have been used relatively herein. However, such terms are not limited to specific coordinate orientations, distances, or sizes, but are used to describe relative positions referencing particular examples. Such terms are not generally limiting to the scope of the claims made herein. Any example or feature of any section, portion, or any other component shown or particularly described in relation to various examples of similar sections, portions, or components herein may be interchangeably applied to any other similar example or feature shown or described herein.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples. Rather these examples should be considered as illustrative and not restrictive in character. All changes and modifications that come within the spirit of the disclosure are to be considered within the scope of the disclosure. The present disclosure should be given the full scope defined by the language of the following claims, and equivalents thereof.

The foregoing description has broad application. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

It should be understood that, as described herein, an “example” (such as illustrated in the accompanying Figures) may refer to an illustrative representation of an environment or article or component in which a disclosed concept or feature may be provided or embodied, or to the representation of a manner in which just the concept or feature may be provided or embodied. However, such illustrated examples are to be understood as one mere example (unless otherwise stated), and other manners of embodying the described concepts or features, such as may be understood by one of ordinary skill in the art upon learning the concepts or features from the present disclosure, are within the scope of the disclosure. In addition, it will be appreciated that while the Figures may show one or more examples of concepts or features together in a single example of an environment, article, or component incorporating such concepts or features, such concepts or features are to be understood (unless otherwise specified) as independent of and separate from one another and are shown together for the sake of convenience and without intent to limit to being present or used together. For instance, features illustrated or described as part of one example can be used separately, or with another example to yield a still further example. Thus, it is intended that the present subject matter covers such modifications and variations as come within the scope of the appended claims and their equivalents.

As used herein, an element or step recited in the singular and proceeded with the word “a” or “an” should be understood as not excluding plural elements or steps, unless such exclusion is explicitly recited.

The phrases “at least one,” “one or more,” and “and/or,” as used herein, are open-ended expressions that are both conjunctive and disjunctive in operation. The terms “a” (or “an”), “one or more” and “at least one” can be used interchangeably herein. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples or configurations. Moreover, the following claims are hereby incorporated into this Detailed Description by this reference, with each claim standing on its own as a separate example of the present disclosure.

We claim:

1. An orthopedic template arranged and configured to assist with removing a previously implanted orthopedic tibial implant during a reversion surgery, the template comprising:
a superior surface and an inferior surface opposite the superior surface, the superior surface including a footprint or representation mimicking a configuration of an underlying support member of the tibial implant.

2. The template of claim 1, wherein the footprint or representation extends superiorly from the superior surface of the template.

3. The template of claim 2, wherein the footprint or representation is a three-dimensional structure extending from the superior surface of the template.

4. The template of claim 3, wherein the footprint or representation is arranged and configured to interact with a first tip of an instrument such that moving the first tip of the instrument relative to the footprint or representation causes a second tip of the instrument to cut bone located inferiorly of the tibial implant.

5. The template of claim 4, wherein the first tip of the instrument has a first length and the second tip of the instrument has a second length smaller than the first length.

6. The template of claim 5, wherein the first and second lengths are configured so that when the first tip contacts the footprint or representation on the template, the second tip approaches but does not contact the support member of the tibial implant.

7. The template of claim 1, wherein the footprint or representation is a printed image that mimics the configuration of the support member of the tibial implant.

8. The template of claim 1, wherein the template is arranged and configured to be positioned on a superior surface of the tibial implant.

9. The template of claim 8, wherein the template is coupled to the tibial implant.

10. The template of claim 1, wherein the template includes an outer shape that mimics an outer shape of the tibial implant.

11. The template of claim 1, wherein the template is a sticker arranged and configured to be adhered to a superior surface of the tibial implant.

12. A system arranged and configured to assist with removing a previously implanted orthopedic tibial implant during a reversion surgery, the tibial implant including a tibial tray and a support member, the tibial tray including a superior surface and an inferior surface, the support member extending inferiorly from the inferior surface of the tibial tray, the system comprising:
an orthopedic template including a superior surface and an inferior surface opposite the superior surface, the template being arranged and configured to be positioned on the superior surface of the tibial tray, the superior surface of the template including a footprint or representation mimicking a configuration of the support member of the tibial implant; and
an instrument including a distal end having first and second tips;
wherein the first tip is arranged and configured to interact with the footprint or representation formed on the superior surface of the template such that moving the first tip of the instrument relative to the footprint or representation causes the second tip to cut bone located inferiorly of the tibial implant.

13. The system of claim 12, wherein the first tip has a first length and the second tip has a second length smaller than the first length.

14. The system of claim 13, wherein the first and second lengths are configured so that when the first tip contacts the footprint or representation on the template, the second tip approaches but does not contact the support member of the tibial implant.

15. The system of claim 12, wherein the footprint or representation extends superiorly from the superior surface of the template.

16. The system of claim 15, wherein the footprint or representation is a three-dimensional structure extending from the superior surface of the template.

17. The system of claim 12, wherein the footprint or representation is a printed image that mimics the configuration of the support member of the tibial implant.

18. A method for removing a tibial implant previously implanted within a patient's bone, the tibial implant including a tibial tray and a support member extending inferiorly of the support member, the support member having a configuration, the method comprising:
positioning an orthopedic template on a superior surface of the tibial implant, the template including a superior surface having a footprint or representation mimicking a configuration of a support member of the tibial implant;
tracing, using a first tip of an instrument, the footprint or representation formed on the superior surface of the template so that a second tip of the instrument cuts bone positioned inferiorly of the tibial tray; and
removing the tibial implant from the patient's bone.
